(19) United States
(12) Patent Application Publication
(10) Pub. No.: US 2018/0164244 A1
(43) Pub. Date: Jun. 14, 2018

(54) CHEMICAL SENSOR

(71) Applicant: Panasonic Corporation, Osaka (JP)

(72) Inventor: HIROAKI OKA, Osaka (JP)

(73) Assignee: Panasonic Corporation, Osaka (JP)

(21) Appl. No.: 15/571,041

(22) PCT Filed: Apr. 28, 2016
(86) PCT No.: PCT/JP2016/002224

§ 371 Date: Nov. 1, 2017

(30) Foreign Application Priority Data
May 15, 2015 (JP) 2015-099579

(51) Int. Cl. G01N 27/414; G01N 33/00; H01L 29/423

(52) U.S. Cl. CPC G01N 27/414; H01L 29/42324; G01N 33/0047

(57) ABSTRACT

A chemical sensor has a field-effect transistor, a detection region provided above the field-effect transistor, and a sensitive film provided in the detection region. The sensitive film includes a metal organic framework.

### TECHNICAL FIELD

The present disclosure relates to chemical sensors capable of detecting chemical molecules such as volatile organic compounds contained in a sample.

### BACKGROUND

As chemical sensors which detect components such as chemical molecules, DNA, or a protein substance contained in a sample, chemical field-effect transistors (ChemFET), ion-selective field-effect transistors (ISFET), etc. are used.

In a conventional chemical field-effect transistor, for example, a sensitive passivation layer is provided on a gate electrode. Chemical molecules, etc. contained in a sample are absorbed by the passivation layer. When the chemical molecules are absorbed by the passivation layer, an amount of a current which flows between a source electrode and a drain electrode of the field-effect transistor is changed. As a result, the chemical field-effect transistor can detect the chemical molecules.

Note that examples of technical literature related to the present disclosure include Patent Literatures 1 to 3.

### CITATION LIST

### Patent Literature

PTL 1: Unexamined Japanese Patent Publication No. 2013-64746

PTL 2: Unexamined Japanese Patent Publication No. H03(1991)-21063

PTL 3: Unexamined Japanese Patent Publication No. 2012-159511

### SUMMARY

A chemical sensor of the present disclosure has a field-effect transistor, a detection region provided above the field-effect transistor, and a sensitive film provided in the detection region. The sensitive film includes a metal organic framework.

The chemical sensor of the present disclosure can accurately detect a detection target component in a sample.

### BRIEF DESCRIPTION OF DRAWINGS

FIG. 1 is a perspective view illustrating a chemical sensor according to an exemplary embodiment.

FIG. 2 is a cross-sectional view illustrating a chemical sensor according to the exemplary embodiment.

FIG. 3 is a cross-sectional view illustrating another example of the chemical sensor according to the exemplary embodiment.

FIG. 4 is a perspective view illustrating a first modified example of the chemical sensor according to the exemplary embodiment.

FIG. 5 is a cross-sectional view illustrating a second modified example of the chemical sensor according to the exemplary embodiment.

### DESCRIPTION OF EMBODIMENT

Prior to describing an exemplary embodiment of the present disclosure, a problem in conventional techniques will be briefly described. Conventional chemical sensors do not have sufficient selectivity of chemical molecules contained in a sample. Therefore, the conventional chemical sensors have a problem that a detection target component in a sample cannot be accurately detected. The present disclosure provides a chemical sensor that solves the above described problem and is capable of accurately detecting a detection target component contained in a sample.

Hereinafter, a chemical sensor according to an exemplary embodiment of the present disclosure will be described in detail with reference to drawings. It should be noted that the exemplary embodiment described below illustrates one specific preferred example of the present disclosure. Therefore, values, shapes, materials, constituent elements, disposition and connection forms of the constituent elements, etc. described in the following exemplary embodiment are examples and are not intended to limit the present disclosure. Accordingly, among the constituent elements in the following exemplary embodiment, a constituent element that is not described in an independent claim indicating a most superordinate concept of the present invention is described as an optional constituent element.

In addition, the drawings are schematic views that do not always illustrate exactly. In the drawings, the same reference mark is applied to the substantially same structure, and redundant description is omitted or simplified.

### EXEMPLARY EMBODIMENT

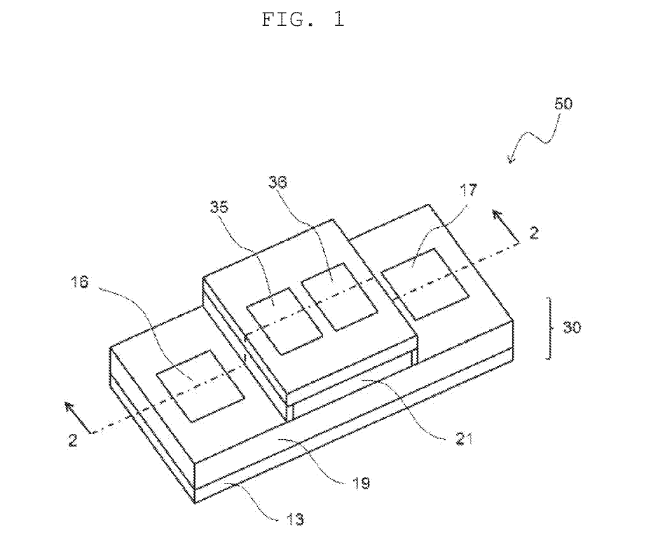

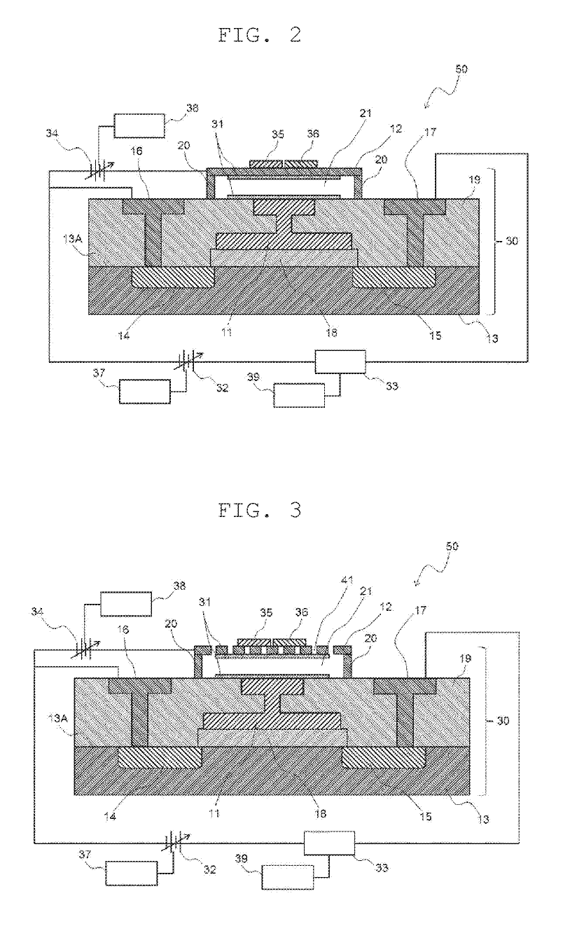

FIG. 1 is a perspective view schematically illustrating chemical sensor 50 according to a present exemplary embodiment. FIG. 2 is a cross-sectional view schematically illustrating a cross section taken along line 2-2 of chemical sensor 50 in FIG. 1.

Chemical sensor 50 detects detection target components such as chemical molecules, nucleic acids, or peptides contained in a sample. The chemical molecules are, for example, volatile organic compounds. The sample is, for example, a breath or blood of a human or an animal. Note that the sample is not limited to a sample derived from a living body, but the sample may be an exhaust gas, air, or the like.

Examples of the volatile organic compound include ketones, amines, alcohols, aromatic hydrocarbons, aldehydes, esters, organic acids, hydrogen sulfide, methyl mercaptan, and disulfides. The volatile organic compounds may be alkanes, alkenes, alkynes, dienes, alicyclic hydrocarbons, allenes, ethers, carbonyls, carb anions, peptides, polynuclear aromatics, heterocyclic rings, organic derivatives, biological molecules, metabolites, isoprene, isoprenoids, and derivatives thereof.

A molecular weight of the volatile organic compounds is preferably between 15 and 500, inclusive, and is more preferably between 30 and 400, inclusive, from a viewpoint of volatility.

According to the World Health Organization (WHO), the volatile organic compounds in a broad sense are categorized into very volatile organic compounds (VVOC, boiling temperature: 0° C. to 50° C.-100° C.), volatile organic compounds (VOC, boiling temperature: 50° C.-100° C. to 240° C.-260° C.), semi-volatile organic compounds (SVOC, boiling temperature: 240° C.-260° C. to 380° C.-400° C.), and particulate organic matters (POM, boiling temperature: 380° C. or higher). Typical VVOC is formaldehyde (molecular weight: 30, boiling temperature: −19.2° C.), acetaldehyde (molecular weight: 44, boiling temperature 20.2° C.), and dichloromethane (molecular weight: 85, boiling temperature: 40° C.). Typical VOC is toluene (molecular weight: 92, boiling temperature 110.7° C.), xylene (molecular weight: 106, boiling temperature: 144° C.), benzene (molecular weight: 78, boiling temperature: 80.1° C.), styrene (molecular weight: 104, boiling temperature: 145.1° C.), etc. Typical SVOC is tributyl phosphate (molecular weight: 266, boiling temperature: 289° C.), dioctyl phthalate (molecular weight: 391, boiling temperature: 370° C.), etc. In the present disclosure, a simple description “volatile organic compounds” means the volatile organic compounds in the broad sense and include VVOC, VOC, SVOC, and POM.

The boiling temperatures of the volatile organic compounds are preferred to range from −160° C. to 400° C., inclusive.

Chemical sensor 50 is provided with field-effect transistor 30, which has floating-gate electrode 11 and counter electrode 12, and sensitive films 31. Herein, floating-gate electrode 11 means an electrically floating gate electrode.

Field-effect transistor 30 is preferred to be a metal oxide semiconductor field effect transistor (MOSFET).

Field-effect transistor 30 has semiconductor substrate 13 having a p-type doping polarity. Examples of a material of semiconductor substrate 13 include silicon, silicon carbide, gallium arsenide, gallium arsenide phosphide, germanium, and gallium nitride.

On first surface 13A of semiconductor substrate 13, diffusion regions 14, 15 having an n-type doping polarity are formed. Diffusion region 14 is disposed to be separated from diffusion region 15. In field-effect transistor 30, diffusion region 14 is a source. Diffusion region 14 serving as the source is formed, for example, by using a metal silicide. As the metal silicide, for example, a silicide of nickel, cobalt, platinum, or palladium can be used. Source electrode 16 is connected to diffusion region 14. Meanwhile, diffusion region 15 is a drain. Drain electrode 17 is connected to diffusion region 15. Diffusion region 15 serving as the drain may be formed by using the same material as the material of diffusion region 14 serving as the source.

As source electrode 16, for example, an electrically conductive material such as copper, aluminum, titanium, titanium nitride, tungsten, zinc oxide, or tin oxide can be used. As drain electrode 17, the same material as the material of source electrode 16 can be used.

Part of first surface 13A between diffusion region 14 and diffusion region 15 is a gate region. Gate oxide 18 is provided on the gate region. As gate oxide 18, for example, silicon dioxide, silicon nitride, silicon oxynitride, aluminum oxide, or tantalum oxide can be used.

As gate oxide 18, a high-dielectric film (High-k material) may be used. Examples of the usable High-k material include hafnium silicate, nitrogen-added hafnium aluminate, hafnium oxide, yttrium oxide, lanthanum oxide, and dysprosium oxide.

Floating-gate electrode 11 is provided on gate oxide 18. As the material of floating-gate electrode 11, for example, an electrically conductive metal can be used. As floating-gate electrode 11, for example, titanium nitride, aluminum, ruthenium, tungsten, silicon tantalum nitride, gold, or platinum can be used. Floating-gate electrode 11 may be formed by using polysilicon.

From a viewpoint of detection sensitivity with respect to the detection target components, a gate-length-direction length of floating-gate electrode 11 is preferred to range from 1 nm to 500 μm, inclusive. Furthermore, the gate-length-direction length of floating-gate electrode 11 is more preferred to range from 30 nm to 100 μm, inclusive. A gate-width-direction length of floating-gate electrode 11 is preferred to range from 1 nm to 500 μm, inclusive. Furthermore, the gate-width-direction length of floating-gate electrode 11 is more preferred to range from 30 nm to 100 μm, inclusive. Herein, the gate-length direction means a direction along a line connecting source electrode 16 and drain electrode 17, and the gate-width direction is a direction approximately perpendicularly intersecting with the gate-length direction in a plane parallel to first surface 13A.

Field-effect transistor 30 is covered with an insulating layer 19 so that a part (an upper surface) of each of floating-gate electrode 11, source electrode 16, and drain electrode 17 is exposed. Note that insulating layer 19 is formed by, for example, the same material as the material of gate oxide 18.

Counter electrode 12 is provided so as to be opposed to floating-gate electrode 11. A hollow region is provided between floating-gate electrode 11 and counter electrode 12.

As a material of counter electrode 12, for example, an electrically conductive metal can be used. Counter electrode 12 may be formed of titanium nitride, aluminum, ruthenium, tungsten, silicon tantalum nitride, gold, or platinum. Counter electrode 12 may use the same material as the material of floating-gate electrode 11.

A distance between floating-gate electrode 11 and counter electrode 12 is preferred to range from 10 nm to 10 μm, inclusive. The distance between floating-gate electrode 11 and counter electrode 12 is more preferred to range from 10 nm to 500 nm, inclusive.

From a viewpoint of detection sensitivity with respect to the detection target components, a gate-length-direction length of counter electrode 12 is preferred to range from 1 nm to 500 μm, inclusive. Furthermore, the gate-length-direction length of counter electrode 12 is more preferred to range from 30 nm to 100 μm, inclusive. A gate-width-direction length of counter electrode 12 is preferred to range from 1 nm to 500 μm, inclusive. Furthermore, the gate-width-direction length of counter electrode 12 is more preferred to range from 30 nm to 100 μm, inclusive. The gate-length-direction length of counter electrode 12 is preferred to be larger than a gate length of floating-gate electrode 11. The gate-width-direction length of counter electrode 12 is preferred to be larger than the gate-width-direction length of floating-gate electrode 11. An area of counter electrode 12 is preferred to be larger than an area of floating-gate electrode 11.

Lateral wall 20 is formed between floating-gate electrode 11 and counter electrode 12. A region surrounded by floating-gate electrode 11, counter electrode 12, and lateral wall 20 is detection region 21. The sample is caused to fill detection region 21. Detection region 21 is preferred to be, for example, a part of a flow channel through which the sample flows. Note that lateral wall 20 may be a part of counter electrode 12.

Sensitive films 31 are provided in detection region 21. Sensitive films 31 are provided on an upper surface of floating-gate electrode 11 and a lower surface of counter electrode 12.

Note that detection region 21 may be provided to be extended from an upper part of floating-gate electrode 11.

Sensitive films 31 capture the detection target components contained in the sample. Sensitive films 31 contain metal organic frameworks (MOF).

The metal organic frameworks are materials having porous structures having high specific surface areas. The porous structures are formed by interactions of metal ions and organic ligands. Since adjustment can be made to provide pore sizes suitable for capturing the detection target components, the metal organic frameworks selectively have high affinity in capturing of the detection target components such as chemical molecules.

Almost all metal ions can be used for the metal organic frameworks. Therefore, the metal ions can impart functions such as magnetism, electric conductivity, thermal conductivity, catalyst characteristics, dielectric characteristics, oxidation-reduction characteristics, optical physicality, and chemical reactivity to the metal organic frameworks. Moreover, functions such as asymmetric points, hydrophobicity/hydrophilicity, optical responsivity, chemical connectivity, and chemical reactivity can be imparted to the metal organic frameworks by designed organic ligands.

In this manner, the metal organic frameworks can be adjusted so as to have the affinity suitable for capturing the detection target components. Therefore, the metal organic frameworks can selectively capture the detection target components such as chemical molecules. Therefore, chemical sensor 50 can accurately detect the detection target components.

As the metal organic frameworks, for example, MOF801-P, MOF801-SC, MOF-802, UiO-66, MOF-808, MOF-841, DUT-67, PIZOF-2, MOF-804, MOF-805, MOF-806, MOF-812, MOF-5, MOF-177, HKUST-1, MIL-53, MIL-96, MIL-101, MAMS-1, Pt/Y MOF, MIL-47, ZMOF-Rho, Dy-btc, Ln-pda, Mn-formata, IRMOF-3, IRMOF-8, IRMOF-111, Zn-IDC, Pd-pymo, Co/DOBDC, Ni/DOBDC, Al-MIL-110, Ni-bpe, MOF-69C, MOF-144, PCN-5, Pt/Zn-MOF, MIL-53calc, UMCM-1, Tb-MOF-76, Mg/DOBDC, PCN-13, ZIF-95, CUK-1, UMCM-150, UMCM-150A, Zn-bdc-DABCO, Ga-MIL-68, Zr-UiO-66, Ti-MIL-125, Pt/ZIF-8, Mg-MOF-74, Co-MOF-74, Ni-MOF-74, CAU-6, CAU-10, SIM-1, aluminum terephthalate (Basolite (registered trade name) A100), Basolite (registered trade name) A300, copper benzene-1,3,5-tricarboxylate (Basolite (registered trade name) C300), zeolite 13X, MCM-41, BPL carbon, etc. can be used.

The metal organic frameworks may contain any one or more metal ions of metal ions such as Zn²⁺, Co²⁺, Ni²⁺, and Cu²⁺. The organic metal frameworks may contain two or more species of the metal ions. The metal organic frameworks may include organic ligands such as oxygen-donor ligands or nitrogen-donor ligands. The organic ligands may be 1,4-benzenedicarboxylic acid, 4,4′-bipyridyl, imidazole, etc. The organic ligands are preferred to have functional groups which exhibit affinity with the detection target components. The organic ligands may have functional groups such as amino groups, aldehyde groups, hydrocarbon groups, carboxyl groups, hydroxy groups, acyl groups, amide groups, carbonyl groups, imino groups, cyano groups, azo groups, thiol groups, sulfo groups, nitro groups, alkyl groups, vinyl groups, allyl groups, aryl groups, phenyl groups, naphthyl groups, aralkyl groups, benzyl groups, cycloalkyl groups, alkoxy groups, methoxy groups, ethoxy groups, etc. The metal organic frameworks may include two or more species of the organic ligands. The metal ions and the organic ligands may be combined by 1:1 or by a different ratio.

The metal organic frameworks are preferred to capture the detection target components by chemical or physical bonding. The metal organic frameworks are preferred to capture the detection target components by covalent bonding, coordination bonding, ion bonding, hydrogen bonding, van der Waals bonding, intermolecular force.

The pore size of the metal organic frameworks is preferred to range from 0.1 nm to 20 nm, inclusive. Furthermore, the pore size of the metal organic frameworks is more preferred to range from 0.4 nm to 6 nm, inclusive. The pore size of the metal organic frameworks is preferred to be larger than diameters of the detection target components such as chemical molecules. The shapes of the pores of the metal organic frameworks may be bottleneck shapes, straight pipe shapes, horn shapes. The pores of the metal organic frameworks may have flexibility. A BET specific surface area of the metal organic frameworks is preferred to be equal to or more than 500 m²/g. Furthermore, the BET specific surface area of the metal organic frameworks is more preferred to be equal to or more than 6000 m²/g. Note that the BET specific surface area is a specific surface area obtained by a BET method.

The metal organic frameworks can be synthesized by a solution method, a microwave method, an ultrasonic method, a solid-phase mixing method. From viewpoints of synthesis readiness and controllability of crystal sizes, a diffusion method, an agitating method, or a hydrothermal method can be used as the solution method.

Note that, as sensitive films 31, a material other than the metal organic frameworks may be used as long as the material can capture the detection target components. For example, as sensitive films 31, covalent organic frameworks (COF), zeolitic imidazolate frameworks (ZIF), or metal organic polyhedron (MOP) porous coordination polymers (PCP) can be also used.

As sensitive films 31, only the metal organic frameworks may be used. As sensitive films 31, a plurality of species of the metal organic frameworks may be stacked. As sensitive films 31, a plurality of metal organic frameworks, covalent organic frameworks, zeolitic imidazolate frameworks, metal organic polyhedrons, and porous coordination polymers may be combined. As sensitive films 31, a plurality of metal organic frameworks, covalent organic frameworks, zeolitic imidazolate frameworks, metal organic polyhedrons, and porous coordination polymers may be stacked.

Note that sensitive films 31 are only required to be provided in detection region 21. For example, sensitive film 31 may be provided on either the upper surface of floating-gate electrode 11 or the lower surface of counter electrode 12. Sensitive film 31 may be provided on a part of lateral wall 20. Sensitive films 31 provided on the upper surface of floating-gate electrode 11, the lower surface of counter electrode 12, and a part of lateral wall 20 may have the same material, but may have different materials.

From a viewpoint of detection sensitivity, a film thickness of sensitive films 31 is preferred to range from 0.1 nm to 1 μm, inclusive. Furthermore, the film thickness of sensitive films 31 is more preferred to range from 10 nm to 100 nm, inclusive.

Sensitive films 31 may be formed by a casting method, a spin coating method, a sputtering method, a vapor deposition method, a liquid-phase growth method, a vapor-phase growth method, a printing method, a pasting method, an electric-field polymerization method, etc.

A joining layer may be provided between sensitive film 31 and floating-gate electrode 11. A joining layer may be provided between sensitive film 31 and counter electrode 12. Sensitive film 31 is preferred to be formed after counter electrode 12 is formed.

Hereinafter, an operation of chemical sensor 50 will be described.

In chemical sensor 50, source electrode 16 and drain electrode 17 are connected via power source device 32. Measuring device 33 that measures a current flowing between source electrode 16 and drain electrode 17 is connected to chemical sensor 50. Power source device 34 that controls electric charge of floating-gate electrode 11 is connected to counter electrode 12. A signal amplifier may be connected to chemical sensor 50. A negative feedback circuit and a temperature compensation circuit may be connected to chemical sensor 50.

Chemical sensor 50 can control an amount of a current which flows from source electrode 16 to drain electrode 17 by controlling electric charge of floating-gate electrode 11 by power source device 34.

A sample is inserted in detection region 21 of chemical sensor 50. Detection target components contained in the sample are captured by sensitive films 31.

When the detection target components are captured by sensitive films 31, work functions of sensitive films 31 are changed. Therefore, an electric charge amount of floating-gate electrode 11 is changed. When the electric charge amount of floating-gate electrode 11 is changed, a value of a current flowing from source electrode 16 to drain electrode 17 is changed. Therefore, chemical sensor 50 can detect the detection target components by detecting changes in the current value.

Chemical sensor 50 can also detect a concentration, etc. of the detection target components by measuring a changed amount, etc. of the current value.

Note that chemical sensor 50 may detect the detection target components by measuring, for example, a value of a voltage applied to counter electrode 12 when the value of the current flowing from source electrode 16 to drain electrode 17 becomes constant.

Note that chemical sensor 50 may control an electric potential of semiconductor substrate 13. Chemical sensor 50 may ground semiconductor substrate 13. Chemical sensor 50 may be operated by a direct current or may be operated by an alternating current.

Note that, as illustrated in FIG. 3, through hole(s) 41 may be formed in counter electrode 12. One or a plurality of through holes 41 may be formed. When through hole(s) 41 are provided, the sample efficiently fills the hollow region.

In order to efficiently fill detection region 21 with the sample, a flow from outside to inside of detection region 21 may be generated. In order to generate the flow, a pump, an air blower, convection can be used.

In detection of the detection target components, the sample in detection region 21 may be flowing or may be still.

Sensitive films 31 are preferred to be cooled so that more detection target components can be captured. For this purpose, chemical sensor 50 may be provided with cooler 35. In detection of the detection target components, temperatures of sensitive films 31 are preferred to range from −80° C. to 25° C., inclusive. Furthermore, in the detection, the temperatures of sensitive films 31 are more preferred to range from −15° C. to 25° C., inclusive. Cooler 35 is provided, for example, on counter electrode 12.

From a viewpoint of improving selectivity in capturing of the detection target components, sensitive films 31 are preferred to be heated. Chemical sensor 50 may be further provided with heater 36. As the heater, for example, a resistive element heater, an infrared heater, or a carbon heater can be used. In the detection of the detection target components, the temperatures of sensitive films 31 are preferred to range from 40° C. to 500° C., inclusive. Furthermore, in the detection the temperatures are more preferred to range from 40° C. to 200° C., inclusive. Heater 36 is provided, for example, on counter electrode 12.

In order to cool or heat sensitive films 31, a thermoelectric element may be used. The thermoelectric element can easily invert a cooling surface and a heating surface. Therefore, the thermoelectric element has functions as cooler 35 and heater 36.

Chemical sensor 50 may be further provided with a temperature sensor. According to a temperature of the chemical sensor measured by the temperature sensor, chemical sensor 50 can carry out temperature compensation with respect to detection results.

Note that the detection target components captured by sensitive films 31 can be removed by a clean gas not containing the detection target components. By virtue of this, chemical sensor 50 can carry out measurement repeatedly. The clean gas is, for example, a dry nitrogen gas, dry air, or a calibration gas.

The metal organic frameworks are preferred to contain organic ligands which exhibit hydrophobicity in order to efficiently detect a sample containing moisture. Before detection target components are detected, the moisture may be removed from the sample. Chemical sensor 50 may be further provided with a humidity sensor.

Before the detection target components are detected, chemical sensor 50 may be initialized. For example, before the detection target components are detected, chemical sensor 50 may be initialized by using the detection target components. Before the detection target components are detected, chemical sensor 50 may be initialized by increasing the temperature of chemical sensor 50.

Furthermore, chemical sensor 50 may be provided with controllers 37, 38 for electrically or mechanically controlling chemical sensor 50. Controller 37 is connected to power source device 32. Controller 38 is connected to power source device 34.

Chemical sensor 50 may be provided with analyzer 39, which analyzes detected results. Analyzer 39 is, for example, connected to measuring device 33. Chemical sensor 50 may carry out multivariable analysis such as principal component analysis, absolute-value representation analysis, discrimination analysis, factor analysis, cluster analysis, or conjoint analysis or may carry out other statistical analysis such as multiple recurrence analysis.

Chemical sensor 50 may be provided with an electromagnetic shield. When the electromagnetic shield is provided, electromagnetic noise from outside can be shielded. Therefore, chemical sensor 50 can efficiently detect minute signals.

Chemical sensor 50 can efficiently detect the detection target components in the sample by providing sensitive films 31 containing the metal organic frameworks.

Moreover, since floating-gate electrode 11 is used as a gate electrode, changes in the work function of the detection region caused by capturing of the detection target components can be accurately detected.

### First Modified Example

Hereinafter, a first modified example of the present exemplary embodiment will be described.

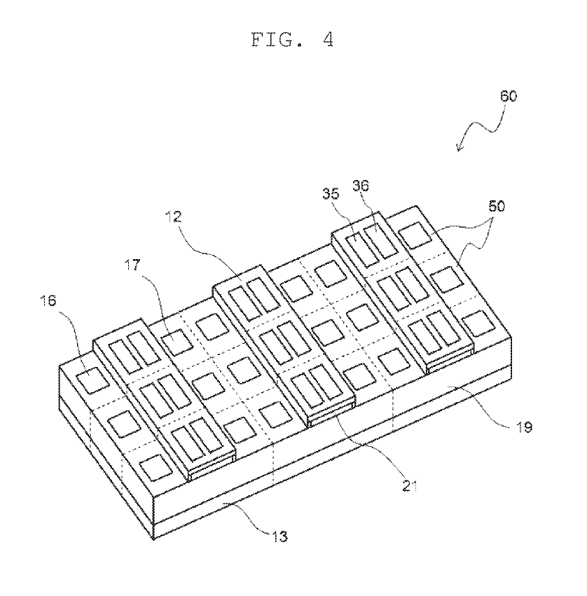

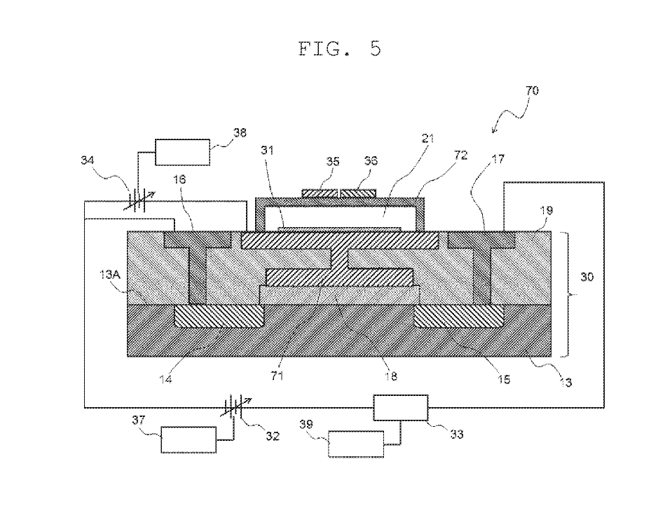

FIG. 4 is a perspective view schematically illustrating chemical sensor 60 according to the first modified example of the present exemplary embodiment.

Chemical sensor 60 of the present modified example has a configuration in which a plurality of chemical sensors 50 according to the present exemplary embodiment is combined. Other configurations are similar to those of chemical sensor 50 described in the exemplary embodiment. The same configurations as those of the exemplary embodiment are denoted with the same reference marks, and description thereof will be omitted.

In chemical sensor 60, the plurality of chemical sensors 50 is provided on single semiconductor substrate 13. A single region sectioned by dotted lines represents single chemical sensor 50.

In other words, chemical sensor 60 is provided with a plurality of field-effect transistors 30 formed on single semiconductor substrate 13. Laterally arranged chemical sensors 50 have detection regions 21 connected to each other. By virtue of this, flow channels through which a sample flows are formed in chemical sensor 60.

Cooler 35 and heater 36 are separately provided in each of chemical sensors 50. Note that coolers 35 and heaters 36 may be connected to each other between laterally arranged chemical sensors 50.

By providing the plurality of chemical sensors 50 on single semiconductor substrate 13 in this manner, chemical sensor 60 can be downsized.

Chemical sensors 50 may be configured by one dimensionally, two dimensionally, or three dimensionally disposing the plurality of field-effect transistors 30. Chemical sensors 50 may be configured by disposing the plurality of field-effect transistors linearly, in a lattice shape, concentrically, radially, or randomly. Chemical sensors 50 may be configured by disposing the plurality of field-effect transistors 30 in parallel to the flow of the sample or disposing the plurality of field-effect transistors 30 at an angle with respect to the flow of the sample.

Chemical sensors 50 may include a plurality of sensitive films 31 in each of the plurality of field-effect transistors 30. Chemical sensors 50 may include sensitive films 31, which are formed by the same material, in each of the plurality of field-effect transistors 30 or may include sensitive films 31 which are formed by different materials.

When sensitive films 31 for the same species are provided in each of chemical sensors 50 in this manner, chemical sensor 60 can provide improved detection accuracy of the detection target components. When sensitive films 31 for different species are provided in each of chemical sensors 50, chemical sensor 60 can detect a plurality of species of detection target components at the same time.

### Second Modified Example

Hereinafter, a second modified example of the present exemplary embodiment will be described.

FIG. 5 is a cross-sectional view schematically illustrating chemical sensor 70 according to the second modified example of the present exemplary embodiment.

Chemical sensor 70 of the present modified example is different from chemical sensor 50 according to the present exemplary embodiment in a point that gate electrode 71 is provided instead of floating-gate electrode 11. Other configurations and operations are similar to those of the exemplary embodiment. The same configurations as those of the exemplary embodiment are denoted with the same reference marks, and description thereof will be omitted.

In field-effect transistor 30, gate electrode 71 is provided on gate oxide 18. Above gate electrode 71, frame body 72 is formed so as to form detection region 21. Frame body 72 is formed by resin or the like. In this case, frame body 72 is provided so that a part of gate electrode 71 is exposed from frame body 72.

Sensitive film 31 is provided on gate electrode 71.

Gate electrode 71 is connected to power source device 34. Power source device 34 controls a voltage applied to gate electrode 71.

Chemical sensor 70 can detect the detection target components by measuring changes in the value of the current flowing from source electrode 16 to drain electrode 17.

Note that chemical sensor 70 may detect the detection target components by measuring, for example, a voltage value applied to gate electrode 71 when the value of the current flowing from source electrode 16 to drain electrode 17 becomes constant. Chemical sensor 70 may detect the detection target components by measuring a change in a threshold voltage.

Note that frame body 72 may have a through hole(s).

A basic operation method and configurations of chemical sensor 70 may be similar to the method and configuration of other chemical sensor 50, 60 described in the exemplary embodiment.

In each of chemical sensors 50, 60, 70 of the present disclosure, a p-type field-effect transistor is used as field-effect transistors 30, but field-effect transistor 30 is not limited thereto. For example, as field-effect transistor 30, an n-type field-effect transistor may be used.

Also, field-effect transistor 30 is not limited to MOSFET. For example, field-effect transistor 30 may be an organic thin-film transistor or an electric double layer transistor using ionic liquid. Field-effect transistor 30 may be an enhance type or may be a depression type.

As described above, one or a plurality of forms of chemical sensors have been described based on the exemplary embodiment. The present disclosure, however, is not limited to the exemplary embodiment. A range of the one form or the plurality of forms may include one obtained by applying various modifications that are conceived by a person skilled in the art to the present exemplary embodiment or a form constructed by combining constituent elements in different exemplary embodiments, without departing from a subject matter of the present disclosure.

### INDUSTRIAL APPLICABILITY

The chemical sensors of the present disclosure are useful in detection of chemical molecules such as volatile organic compounds.

### REFERENCE MARKS IN THE DRAWINGS

11: floating-gate electrode
12: counter electrode
13: semiconductor substrate
14, 15: diffusion region
16: source electrode
17: drain electrode
18: gate oxide
19: insulating layer
20: lateral wall
21: detection region
30: field-effect transistor
31: sensitive film
32, 34: power source device
33: measuring device
35: cooler
36: heater
37, 38: controller
39: analyzer
41: through hole
50, 60, 70: chemical sensor
71: gate electrode
72: frame body

1. A chemical sensor comprising:
a field-effect transistor;
a detection region provided above the field-effect transistor; and
a sensitive film provided in the detection region,
wherein the sensitive film includes a metal organic framework.

2. The chemical sensor according to claim 1, wherein the field-effect transistor has a floating-gate electrode and a counter electrode opposed to the floating-gate electrode via the detection region.

3. The chemical sensor according to claim 1, wherein the field-effect transistor has a gate electrode and a frame body provided so as to form the detection region above the gate electrode.

4. The chemical sensor according to claim 1, wherein the metal organic framework includes at least one of metal ions selected from the group consisting of Zn2+, Co2+, Ni2+, and Cu2+.

5. The chemical sensor according to claim 1, wherein the metal organic framework is constituted by an organic ligand of an oxygen-donor ligand or a nitrogen-donor ligand.

6. The chemical sensor according to claim 1, wherein the metal organic framework is any one selected from the group consisting of MOF801-P, MOF801-SC, MOF-802, UiO-66, MOF-808, MOF-841, DUT-67, PIZOF-2, MOF-804, MOF-805, MOF-806, MOF-812, MOF-5, MOF-177, HKUST-1, MIL-53, MIL-96, MIL-101, MAMS-1, Pt/Y MOF, MIL-47, ZMOF-Rho, Dy-btc, Ln-pda, Mn-formata, IRMOF-3, IRMOF-8, IRMOF-111, Pd-pymo, Co/DOBDC, Ni/DOBDC, Al-MIL-110, Ni-bpe, MOF-69C, MOF-144, PCN-5, Pt/Zn-MOF, MIL-53calc, UMCM-1, Tb-MOF-76, Mg/DOBDC, PCN-13, ZIF-95, CUK-1, UMCM-150, UMCM-150A, Zn-bdc-DABCO, Ga-MIL-68, Zr-UiO-66, Ti-MIL-125, Pt/ZIF-8, Mg-MOF-74, Co-MOF-74, Ni-MOF-74, CAU-6, CAU-10, SIM-1, aluminum terephthalate, copper benzene-1,3,5-tricarboxylate, zeolite 13X, MCM-41, and BPL carbon.

7. The chemical sensor according to claim 2, wherein the counter electrode has a through hole.
